(19) United States
(12) Patent Application Publication
(10) Pub. No.: US 2009/0182256 A1
(43) Pub. Date: Jul. 16, 2009

(54) Removable protection adhesive tape structure with easily tearing-off feature

(76) Inventor: Chung-Yu Lin, Kaohsiung City (TW)

Correspondence Address: BACON & THOMAS, PLLC, 625 SLATERS LANE, FOURTH FLOOR, ALEXANDRIA, VA, 22314-1176, US

(21) Appl. No.: 12/318,366

(22) Filed: Dec. 29, 2008

Related U.S. Application Data
(63) Continuation-in-part of application No. 11/652,630, filed on Jan. 12, 2007, now abandoned.

(51) Int. Cl. A61F 13/00

(52) U.S. Cl. 602/54

(57) ABSTRACT

A removable medical adhesive tape with easily tearing-off feature of continual enrolled flat porous non-woven fabric strip in roll type includes a front layer, a backing layer, a pair of short transverse ends and a pair of prolonged longitudinal lateral edges in symmetrical parallel juxtaposition. Wherein a plurality of lengthwise intermediate adhesive bands intervenes between the pair of prolonged longitudinal lateral edges in parallel spaced manner; a lengthwise non-adhesive band is formed between each pair of adjacent intermediate adhesive bands respectively so that a plurality of non-adhesive cutouts are created at the freely exposed short transverse end; and the non-woven fabric strip is made of plural transverse textures and longitudinal textures in cross-linked manner such that the transverse texture is made into having highly extensible and elastic stretchability whereas the longitudinal texture is made into no stretchability.

This is a Continuation-In-Part Application of Non provisional application, application Ser. No. 11/652,630, field Jan. 12, 2007.

### FIELD OF THE PRESENT INVENTION

The present invention relates to a removable medical adhesive tape with easily tearing-off feature for using in household labor services or physical exercises. By adhering beforehand the medical adhesive tape on the joints or muscles, which is possibly overused, the protective effects over the joints or muscles can be achieved.

### BACKGROUND OF THE INVENTION

During daily household labor services or physical exercises, joints or muscles easily become fatigue or sore due to overuse and results in chronic diseases for long time without suitable medical care. Accordingly, various conventional medical bandages or tapes for releasing sore and ache are promoted in the drugstores. These medical bandages or tapes might be able to passively mitigate the suffering to some degree. However, they can not have the preventing effect in advance. Normally, the dressings on the foregoing conventional medical bandages or tapes are blended in the adhesive glue to spread over the coating surface thereof. On using such conventional medical bandages or tapes, the skin of suffering joints or muscles is wrapped with possible susceptibility to the allergy for some people. Moreover, it is always not easy to tear off such conventional medical bandages or tapes from the skin of suffering joints or muscles because of no suitable easily tearing-off feature is contrived therein. Usually on tearing off procedure, a tiny edge or corner of such conventional medical bandages or tapes is first lifted by one finger, then tear off the whole adhesive bandage from the skin by two fingers to finish the tearing off job. Consequently, the skin of suffering joints or muscles is incurred by pain during the tearing off procedure throughout.

To solve foregoing drawback existed in the conventional medical bandages or tapes aforesaid, the U.S. Pat. Nos. 1,707,515 and 2,310,082 disclose a common design therefore that has intermediate non-adhesive area for easily tearing off. However, neither U.S. Pat. No. 1,707,515 nor U.S. Pat. No. 2,310,082 has a feature for protective effect on the joint or muscle.

### SUMMARY OF THE INVENTION

The present invention originates a removable medical adhesive tape with easily tearing-off feature of continual enrolled flat porous non-woven fabric strip in roll type comprising a front layer, a backing layer, a pair of short transverse ends and a pair of prolonged longitudinal lateral edges in symmetrical parallel juxtaposition, wherein a plurality of lengthwise intermediate adhesive bands intervenes between the pair of marginal adhesive bands in parallel spaced manner; and the non-woven fabric strip is made of plural transverse textures and longitudinal textures in cross-linked manner such that the transverse texture is made into having highly extensible and elastic stretchability whereas the longitudinal texture is made into no stretchability. Because of the fixing tension no stretchability in the longitudinal texture of the non-woven fabric strip applied on the dermal surface of movable joints, not only the athletic injure but also the muscular fatigue and sore due to over-exercise on the applied joints can be avoid without sacrifice the freedom of the wrist movement.

Another object of the present invention is to provide a removable medical adhesive tape with easily tearing-off feature that offers a conservational therapy to the human physical portion where is suffered from injure of athletics.

Further object of the present invention is to provide a removable medical adhesive tape with easily tearing-off feature that offers an enhancing effect on the endurance of athletics to the human physical portion where is suffered from muscular fatigue or sore.

Additional object of the present invention is to provide a removable medical adhesive tape with easily tearing-off feature of continual enrolled flat porous non-woven fabric strip in roll type comprises a front layer, a backing layer, a pair of short transverse ends and a pair of prolonged longitudinal lateral edges in symmetrical parallel juxtaposition, wherein one of the pair short transverse ends is exposed for freely unrolling while the other is enrolled in the center of the non-woven fabric strip roll; a lengthwise marginal adhesive band is coated along and flushes with each of the pair longitudinal lateral edges on the front layer of the non-woven fabric strip respectively; a plurality of lengthwise intermediate adhesive bands intervenes between the pair of marginal adhesive bands in parallel spaced manner; and a lengthwise non-adhesive band is formed between each pair of adjacent intermediate adhesive bands and between the marginal adhesive band and the adjacent intermediate adhesive band respectively so that a plurality of non-adhesive cutouts are created at the freely exposed short transverse end to serve as a fulcrum of the fingers during tearing off procedure for mitigating the painful feeling of the skin.

Another object of the present invention is to provide a removable medical adhesive tape with easily tearing-off feature of continual enrolled flat porous non-woven fabric strip in roll type comprising a front layer, a backing layer, a pair of short transverse ends and a pair of prolonged longitudinal lateral edges in symmetrical parallel juxtaposition, wherein a lengthwise non-adhesive band is formed along and flushes with each of the pair longitudinal lateral edges on the front layer of the non-woven fabric strip respectively so that no dirt or dust can be attached on the longitudinal lateral edges.

### BRIEF DESCRIPTION OF THE DRAWINGS

FIG. 1 is a perspective schematic view of a removable medical adhesive tape with easily tearing-off feature according to a first preferred embodiment of the present invention.

FIG. 2 is a partial bottom view of the above first preferred embodiment of the present invention.

FIG. 3 is a sectional view taken along the line 3-3 as indicated in FIG. 1.

FIG. 4 is a sectional view showing the operation of FIG. 3 acted by a transverse pulling force in extending manner.

FIG. 5 is a operational schematic view showing the non-woven fabric strip in the first preferred embodiment of the present invention acted by a transverse pulling force in extending manner.

FIG. 6 is the first schematic view showing the non-woven fabric strip for the first embodiment of the present invention in manner of closely adhering on the wrist back.

FIG. 7 is the second schematic view showing the non-woven fabric strip for the first embodiment of the present invention in manner of closely adhering on the wrist back.

FIG. 8 is the third schematic view showing the non-woven fabric strip for the first embodiment of the present invention in manner of closely adhering on the wrist back.

FIG. 9 is the operational schematic view showing the hand wavers up and down from the wrist joint.

FIG. 10 is the operational schematic view showing the hand, which has the non-woven fabric strip for the first embodiment of the present invention closely adhered on the wrist back, wavers up and down from the wrist joint.

FIG. 11 is the first schematic view showing the non-woven fabric strip for the first embodiment of the present invention in manner of closely adhering on both of the wrist sides for wrist protection.

FIG. 12 is the second schematic view showing the non-woven fabric strip for the first embodiment of the present invention in manner of closely adhering on both of the wrist sides for wrist protection.

FIG. 13 is the schematic view showing the non-woven fabric strip for the first embodiment of the present invention in manner of closely adhering on the finger for finger protection.

FIG. 14 is the schematic view showing the non-woven fabric strip for the first embodiment of the present invention in manner of closely adhering on the hand for DeQuervain's disease prevention.

FIG. 15 is the schematic view showing the non-woven fabric strip for the first embodiment of the present invention in manner of closely adhering on the elbow for golf elbow prevention.

FIG. 16 is the schematic view showing the non-woven fabric strip for the first embodiment of the present invention in manner of closely adhering on the elbow for tennis elbow prevention.

FIG. 17 is the schematic view showing the non-woven fabric strip for the first embodiment of the present invention in manner of closely adhering on the outer lateral of the knee for lateral knee ligament prevention.

FIG. 18 is the first schematic view showing the non-woven fabric strip for the first embodiment of the present invention in manner of closely adhering on the ankle for ankle prevention.

FIG. 19 is the second schematic view showing the non-woven fabric strip for the first embodiment of the present invention in manner of closely adhering on the ankle for ankle prevention.

FIG. 20 is the schematic view showing the non-woven fabric strip for the first embodiment of the present invention in manner of closely adhering on the sole for plantar protection.

FIG. 21 is the operational schematic view showing the non-woven fabric strip for the first embodiment of the present invention in manner of tearing-off action from the wrist back.

FIG. 22 is the schematic view showing the second preferred exemplary embodiment for the non-woven fabric strip of the present invention.

FIG. 23 is a sectional view taken along the line 23-23 as indicated in FIG. 22.

FIG. 24 is the schematic view showing the third preferred embodiment for the non-woven fabric strip of the present invention.

FIG. 25 is a sectional view taken along the line 25-25 as indicated in FIG. 24.

### DETAILED DESCRIPTION OF THE PREFERRED EMBODIMENTS

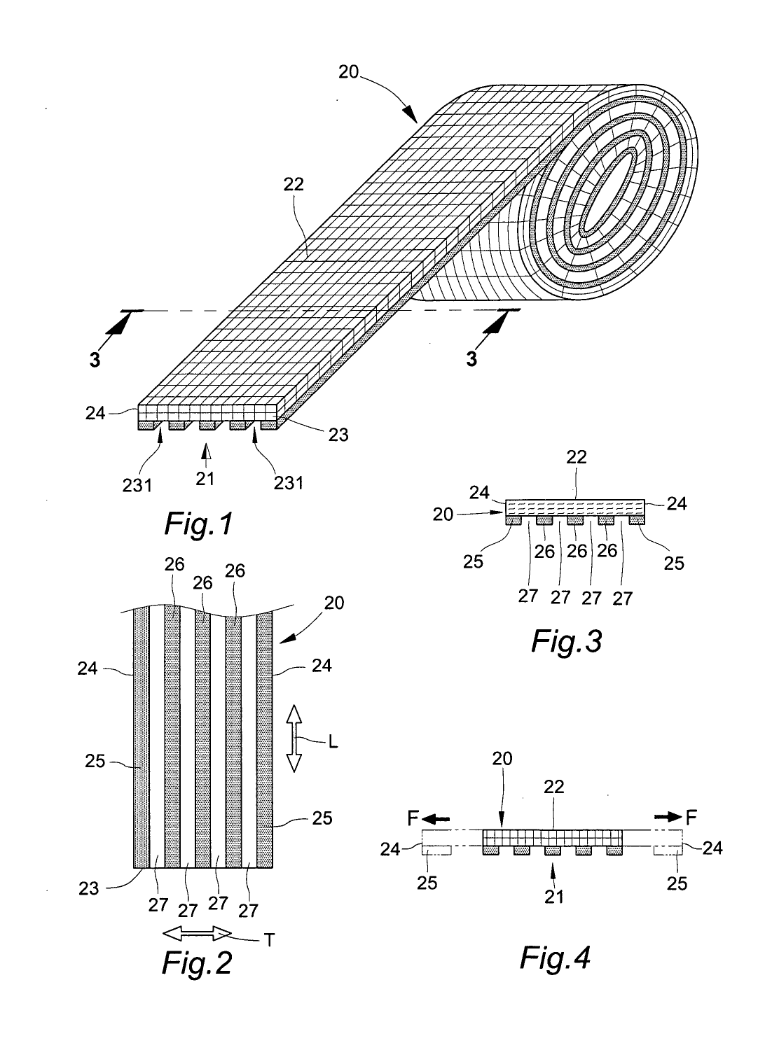

Referring to FIGS. 1 to 5, a removable medical adhesive tape with easily tearing-off feature according to a first preferred embodiment of the present invention. The medical adhesive tape is made of a continual enrolled flat porous non-woven fabric strip 20 in roll type comprising a front layer 21, a backing layer 22, a pair of short transverse ends 23 and a pair of prolonged longitudinal lateral edges 24 in symmetrical parallel juxtaposition, wherein

One of the pair short transverse ends 23 is exposed for freely unrolling while the other is enrolled in the center of the non-woven fabric strip 20 roll (not shown in the figure);

A lengthwise marginal adhesive band 25 is coated along and flushes with each of the pair longitudinal lateral edges 24 on the front layer 21 of the non-woven fabric strip 20 respectively;

A plurality of lengthwise intermediate adhesive bands 26 intervenes between the pair of marginal adhesive bands 25 in parallel spaced manner;

A lengthwise non-adhesive band 27 is formed between each pair of adjacent intermediate adhesive bands 26 and between the marginal adhesive band 25 and the adjacent intermediate adhesive band 26 respectively so that a plurality of non-adhesive indentations 231 are created at the freely exposed short transverse end 23 (as shown in FIG. 1);

Besides, crosswise compositions of the non-woven fabric strip 20, which are perpendicular to the direction of the longitudinal lateral edges 24 as shown of the hollow arrowhead T in FIG. 2, defines the transverse texture while lengthwise compositions of the non-woven fabric strip 20, which are parallel to the direction of the longitudinal lateral edges 24 as shown of the hollow arrowhead L in FIG. 2, defines the longitudinal texture.

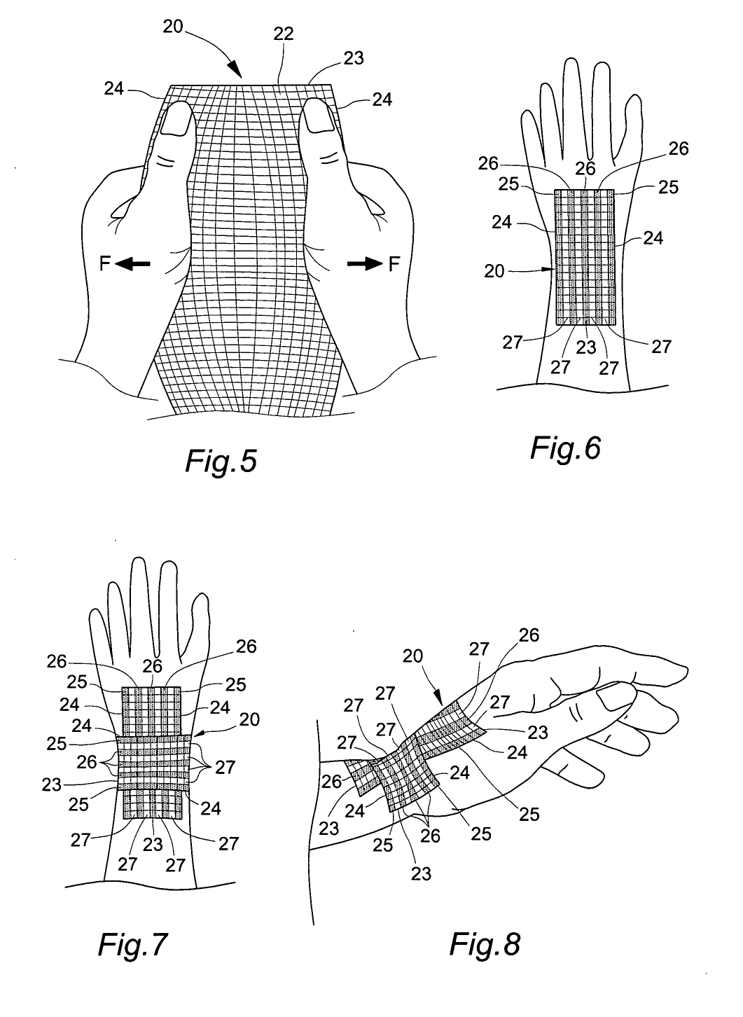

For the first preferred embodiment in the non-woven fabric strip 20, the transverse texture is made into having highly extensible and elastic stretchability in case of being subject to the pulling force F as shown in the arrowhead directions with hypothetic dashed ling in FIG. 4 and hand pulling force F in the arrowhead directions in FIG. 5, whereas the longitudinal texture is made into no stretchability by the pulling force.

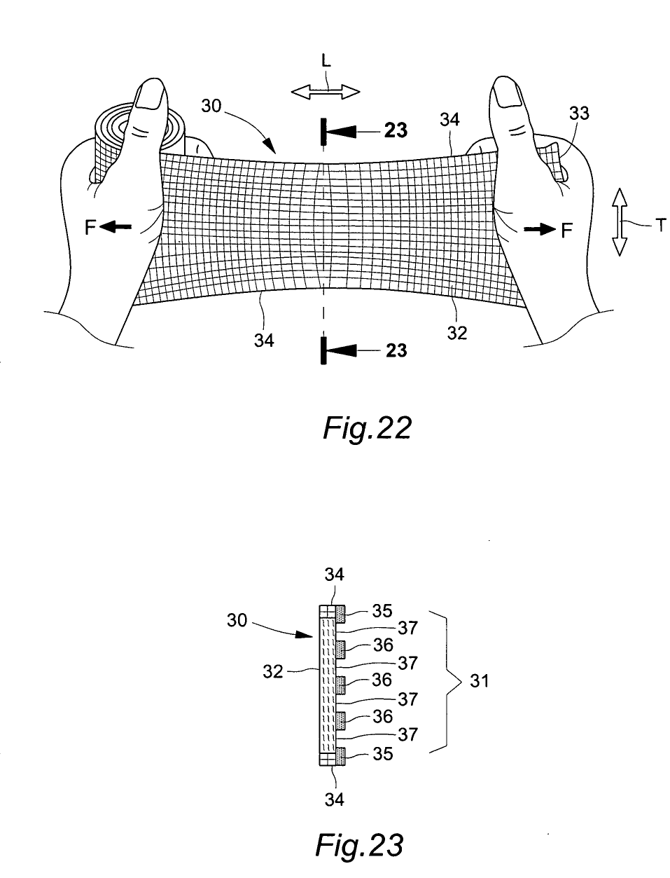

Conversely, for the second preferred embodiment as shown in FIGS. 22 and 23, the longitudinal texture is made into having highly extensible and elastic stretchability, whereas the transverse texture is made into no stretchability.

For practical application of the medical adhesive tape in the first embodiment of the present invention, please refer to FIGS. 6 to 10. As shown in FIGS. 6 through 8, the non-woven fabric strip 20 is closely adhered to the wrist back. Because of no stretchability in the longitudinal texture of the non-woven fabric strip 20, the up-and-down hand wavering action from the wrist joint W in is limited to a certain range due to a fixing tension in non-stretchability of the longitudinal texture applied on the dermal surface of the wrist back. However, the wrist freedom of the leftward or rightward movement is not confined as highly extensible and elastic stretchability in the transverse texture of the non-woven fabric strip 20.

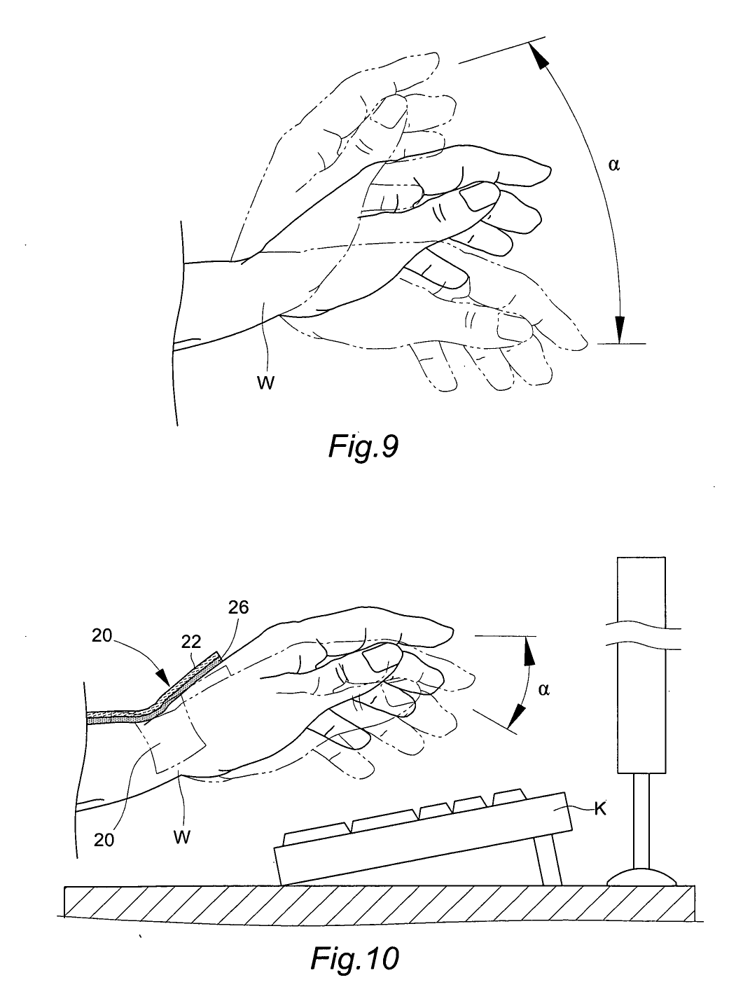

In the daily practical usage, taking the key-in action on the keyboard K of the personal computer (PC) as an example is described as below. As shown in FIG. 9, which has no non-woven adhesive tape of the present invention closely adhered on the wrist back, the hand wavering angle α of up-and-down action from the wrist joint W is free to maximal possibility. For long-term practice, the muscular fatigue or sore due to over-exercise on the wrist joint W is inevitable. Whereas in FIG. 10, which has the non-woven adhesive tape of the present invention closely adhered on the wrist back, the hand wavering angle α of up-and-down action from the wrist joint W is confined to a smaller degree so that the muscular fatigue or sore of the wrist joint W is mitigated to achieve the wrist protection. Therefore, by closely adhering the non-woven adhesive tape for the first exemplary embodiment of the present invention on the wrist back, not only the athletic injure but also the muscular fatigue and sore due to over-exercise on the wrist joint W can be avoid without sacrifice the freedom of the wrist movement.

Moreover, additional piece of non-woven fabric strip 20 can crosswise overlaps the backing layer 22 of the existing lengthwise non-woven fabric strip 20 on the wrist back (as shown in FIGS. 7 and 8) so as to promote the fixing tension so that the wrist protection is enhanced even under long time practice in the key-in action on the keyboard K of the personal computer (PC).

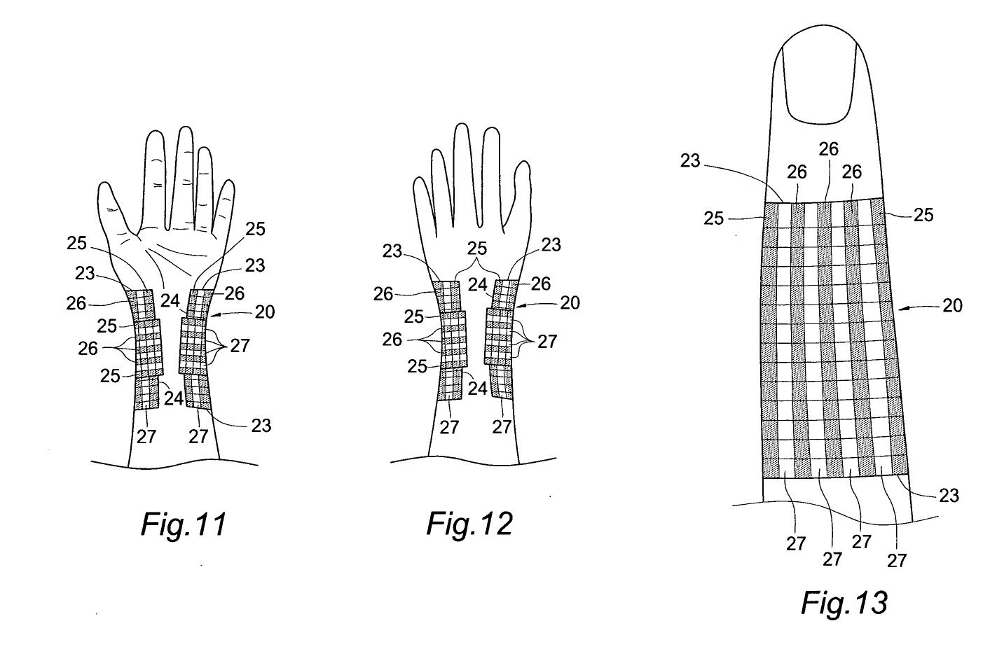

For more practical usage examples of the non-woven adhesive tape for the first exemplary embodiment of the present invention, as shown in FIGS. 11 and 12, plural pieces of the non-woven fabric strip 20 are crosswise adhered on both of the wrist sides for wrist protection to avoid the wrist from muscular fatigue and sore.

As shown in FIG. 13, single piece of the non-woven fabric strip 20 is closely adhered on the finger in longitudinal manner for finger protection to avoid the finger wrist from muscular fatigue and sore.

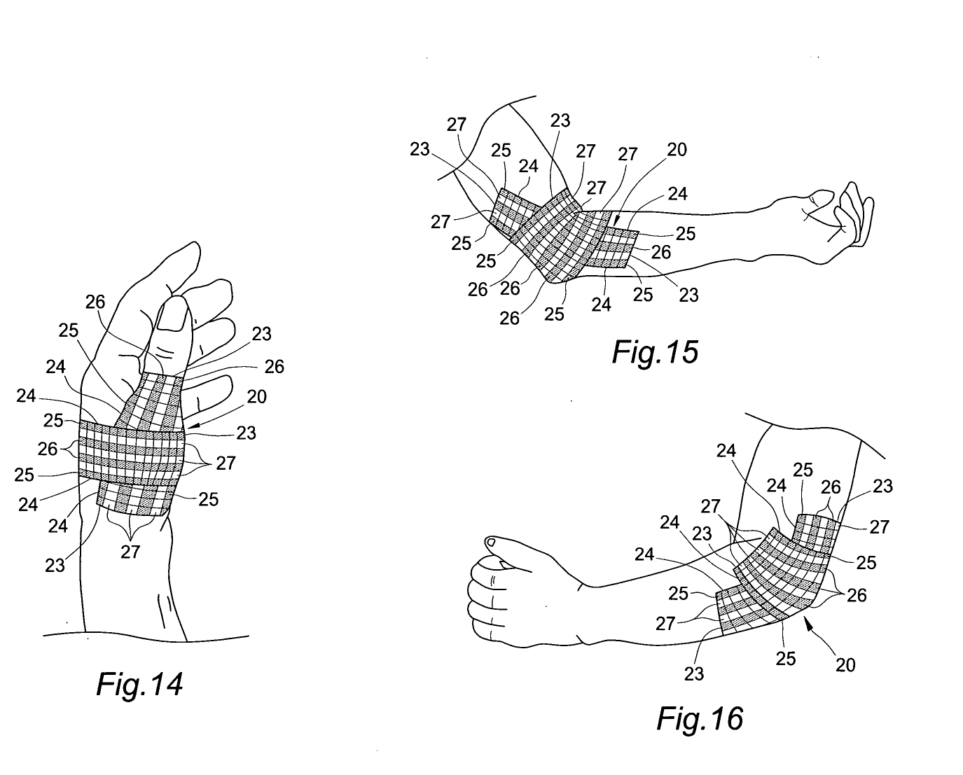

Moreover, as shown in FIG. 14, plural pieces of the non-woven fabric strip 20 are closely adhered on the hand in crosswise manner for DeQuervain's disease prevention to avoid the hand wrist from muscular fatigue and sore.

Further, as shown in FIG. 15, plural pieces of the non-woven fabric strip 20 are closely adhered on the elbow in crosswise manner for golf elbow prevention to avoid the elbow from muscular fatigue and sore.

Similarly, as shown in FIG. 16, plural pieces of the non-woven fabric strip 20 are also closely adhered on the elbow in crosswise manner for tennis elbow prevention to avoid the elbow from muscular fatigue and sore.

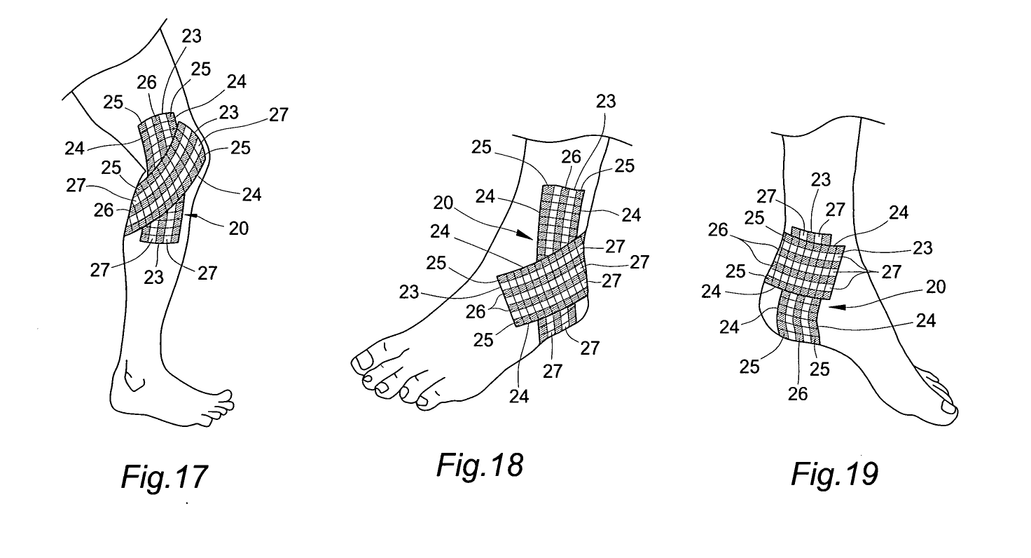

Furthermore, as shown in FIG. 17, plural pieces of the non-woven fabric strip 20 are closely adhered on the outer lateral of the knee in crosswise manner for lateral knee ligament prevention to avoid the lateral knee ligament from muscular fatigue and sore.

Likewise, as shown in FIGS. 18 and 19, plural pieces of the non-woven fabric strip 20 are closely adhered on the ankle in crosswise manner for ankle prevention to avoid the ankle from muscular fatigue and sore.

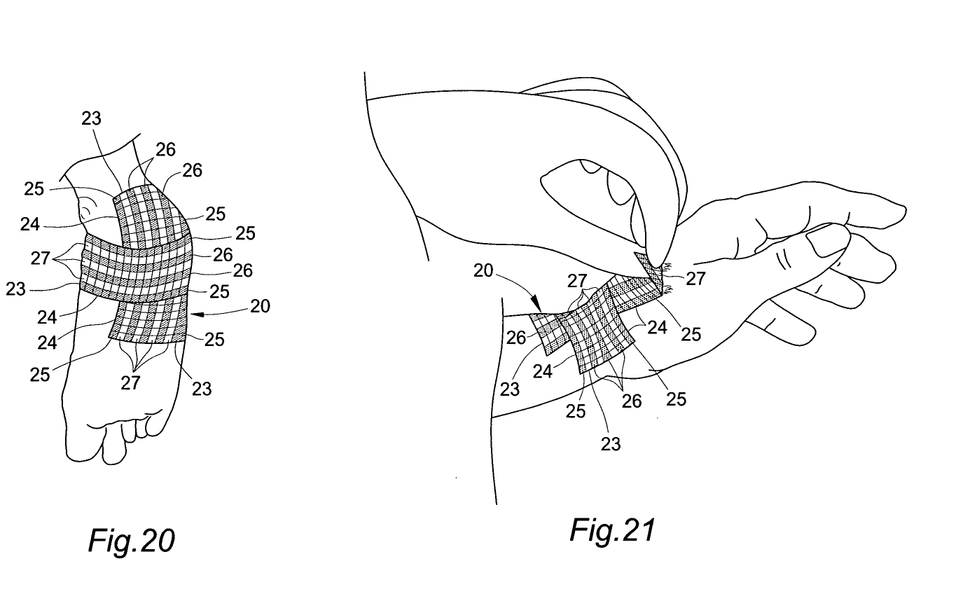

Additionally, as shown in FIG. 20, plural pieces of the non-woven fabric strip 20 are closely adhered on the sole in open ring and U-shape cross manner for plantar protection to avoid the sole from muscular fatigue and sore.

For tearing off or ripping off the non-woven fabric strip 20 from the dermal surface S, taking that on the back of the wrist joint W shown in FIG. 21 as example, just align finger with any non-adhesive indentation 231 of the non-adhesive band 27 on the short transverse end 23 as grip fulcrum for lifting up to achieve the purpose of tearing off the adhesive tape from the dermal surface S quickly without resorting to any tools so that the painful feeling incurred on the skin can be alleviated. Likewise, the same way of tearing off the non-woven fabric strip 20 from the dermal surface S can be also applicable to other portion of the human body, where single or plural pieces of the non-woven fabric strip 20 are adhered as disclosed heretofore. Therefore, redundant description is saved here.

FIGS. 22 and 23 illustrate a second preferred embodiment of the present invention, in which the non-woven fabric strip 30 is similar to the first preferred embodiment and made of a continual enrolled flat porous non-woven fabric strip in roll type comprising a front layer 31, a backing layer 32, a pair of short transverse ends 33 and a pair of prolonged longitudinal lateral edges 34 in symmetrical parallel juxtaposition, wherein a lengthwise marginal adhesive band 35 is coated along and flushes with each of the pair longitudinal lateral edges 34 on the front layer 31 of the non-woven fabric strip 30 respectively; A plurality of lengthwise intermediate adhesive bands 36 intervenes between the pair of marginal adhesive bands 35 in parallel spaced manner; A lengthwise non-adhesive band 37 is formed between each pair of adjacent intermediate adhesive bands 36 and between the marginal adhesive band 35 and the adjacent intermediate adhesive band 36 respectively.

Besides, crosswise compositions of the non-woven fabric strip 30, which are perpendicular to the direction of the longitudinal lateral edges 34 as shown of the hollow arrowhead T in FIG. 22, defines the transverse texture while lengthwise compositions of the non-woven fabric strip 30, which are parallel to the direction of the longitudinal lateral edges 34 as shown of the hollow arrowhead L in FIG. 22, defines the longitudinal texture.

On the contrary to the non-woven fabric strip 20 in the first preferred embodiment of the present invention. The non-woven fabric strip 30 in the second preferred embodiment of the present invention, the longitudinal texture is made into having highly extensible and elastic stretchability in case of being subject to the pulling force F as the hand pulling force F in the arrowhead directions shown in FIG. 22, whereas the transverse texture is made into no stretchability by the pulling force.

By combinational use the different non-woven fabric strips 20 & 30 of the first and second preferred embodiments, the application versatility and adaptability for the non-woven adhesive tape of the present invention can be substantially enhanced to further achieve the effects in avoidance the athletic injure as well as the muscular fatigue and sore to desirable extend.

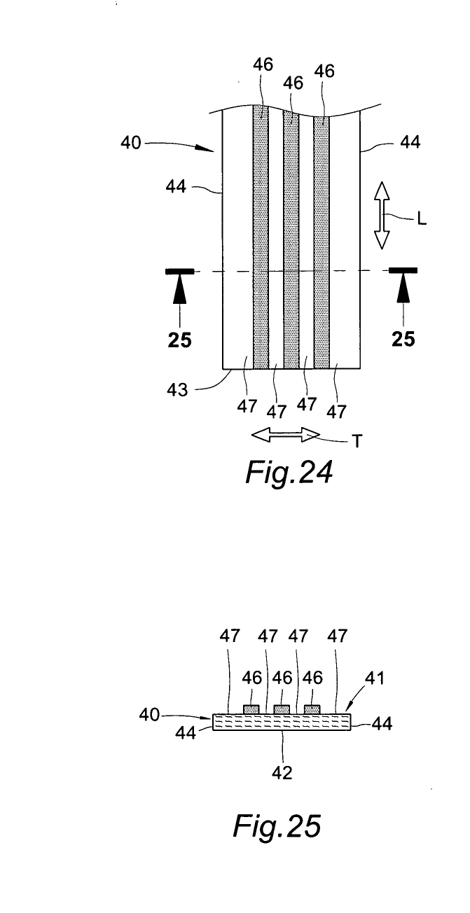

Besides, the non-woven adhesive tape shown in FIGS. 24 and 25 is the third preferred embodiment for the medical adhesive tape of the present invention. The medical adhesive tape is made of a continual enrolled flat porous non-woven fabric strip 40 in roll type comprising a front layer 41, a backing layer 42, a pair of short transverse ends 43 and a pair of prolonged longitudinal lateral edges 44 in symmetrical parallel juxtaposition, wherein a lengthwise non-adhesive band 47 is formed along and flushes with each of the pair longitudinal lateral edges 44 on the front layer 41 of the non-woven fabric strip 40 respectively so that no dirt or dust can be attached on the longitudinal lateral edges 44.

In conclusion, because the extensible and elastic stretchability to the longitudinal texture and the transverse texture in the porous non-woven fabric strip for the medical adhesive of the present invention is adaptable to combine at will, the non-woven medical adhesive tape of the present invention can be used in adhering it to the different vulnerable parts of the human body such as wrist, dorsal wrist, DeQuervain's disease, golf elbow, tennis elbow, tennis elbow, lateral/medial knee ligament, ankle, plantar and the like to serve as soft flexible splint for supporting and reinforcing effect in consequence of the physical prevention/protection so that multiple effects in avoiding injure of athletics, conservational therapy in injure of athletics, and enhance on the endurance of athletics can become achievable.

What is claimed is:

1. A removable medical adhesive tape with easily tearing-off feature of continual enrolled flat porous non-woven fabric strip in roll type comprises a front layer, a backing layer, a pair of short transverse ends and a pair of prolonged longitudinal lateral edges in symmetrical parallel juxtaposition, wherein
One of the pair short transverse ends is exposed for freely unrolling while the other is enrolled in the center of said non-woven fabric strip roll;
A lengthwise marginal adhesive band is coated along and flushes with each of the pair longitudinal lateral edges on said front layer of said non-woven fabric strip respectively;
A plurality of lengthwise intermediate adhesive bands intervenes between the pair of marginal adhesive bands in parallel spaced manner;
A lengthwise non-adhesive band is formed between each pair of adjacent intermediate adhesive bands and between said marginal adhesive band and said adjacent intermediate adhesive band respectively so that a plurality of non-adhesive indentation are created at the freely exposed short transverse end; and
Said non-woven fabric strip is made of plural transverse textures and longitudinal textures in cross-linked manner such that the transverse texture is made into having highly extensible and elastic stretchability whereas the longitudinal texture is made into no stretchability.

2. A removable medical adhesive tape with easily tearing-off feature, as recited in claim 1, wherein said longitudinal texture is made into having highly extensible and elastic stretchability, whereas the transverse texture is made into no stretchability.

3. A removable medical adhesive tape with easily tearing-off feature, as recited in claim 1, wherein each said lengthwise marginal adhesive bands is replaced by a lengthwise non-adhesive band respectively.
